(19) United States
(12) Patent Application Publication
(10) Pub. No.: US 2012/0270300 A1
(43) Pub. Date: Oct. 25, 2012

(54) PRODUCTION OF RECOMBINANT FACTOR IX IN A HUMAN HEPATOCYTE CELL LINE

(75) Inventors: Nathalie Enjolras, Lyon (FR); Claude Negrier, Irigny (FR); Yesim Dargaud, Lyon (FR)

(73) Assignee: Universite Claude Bernard Lyon I, Villeurbanne Cedex (FR)

(21) Appl. No.: 13/505,936

(22) PCT Filed: Nov. 4, 2010
(86) PCT No.: PCT/FR10/52377

§ 371 Date: Jul. 11, 2012

(30) Foreign Application Priority Data
Nov. 6, 2009 (FR) 0957890

(51) Int. Cl. C12N 9/64; C12N 5/10

(52) U.S. Cl. 435/226; 435/370

(57) ABSTRACT

The invention relates to a recombinant human factor IX (FIX) characterized in that said factor is obtained by a preparation method comprising, or even consisting of, the steps which consist in causing the genetic material encoding the FIX to be expressed in vitro in a human hepatocyte cell line Huh7, recovering the cellular supernatant in which the FIX was secreted and, optionally, purifying the synthesized FIX.

The present invention relates to the field of biology, and in particular the production of recombinant proteins.

More specifically, the subject of the present invention is essentially a novel hepatocyte cell line capable of producing biologically active recombinant factor IX, and also the recombinant factor IX thus produced.

Human factor IX (FIX) is a protein of 415 amino acids (mature form) naturally present in the blood which participates in the cascade of reactions resulting in blood coagulation.

FIX is mainly synthesized by the liver, in the form of a precursor which, after removal of the signal peptide, is subjected to various post-translational modifications such as gamma-carboxylation of glutamic acids, glycosylation of asparagines, of serines and of threonines, beta-hydroxylation of an aspartic acid, phosphorylation of a serine, or sulfation of a tyrosine. After removal of the propeptide before tyrosine 1, the active FIX is then secreted into the blood stream.

The importance of these post-translational modifications (and of the removal of the propeptide) in obtaining a biologically active FIX has been demonstrated by several authors (Pipe, Thromb Haemost. 2008 May; 99(5): 840-50).

Hemophilia is the most common of the serious hemorrhagic diseases. It is a recessive genetic disease, the transmission of which is linked to the X chromosome. A deficiency in coagulation factor VIII (FVIII) characterizes hemophilia A (one birth in 5000) and a deficiency in coagulation factor IX characterizes hemophilia B (one birth in 30 000). Clinical expressions of hemophilia can be classified according to the level of the factor concerned, namely the severe forms (FVIII/FIX<1%), moderate forms (FVIII/FIX from 1-5%) and minor forms (FVIII/FIX>5%).

The treatment for hemophilia (severe or moderate) consists, of a replacement treatment by administering FVIII/FIX concentrates, on demand, during hemorrhagic events or surgical procedures and/or as prophylaxis for preventing hemorrhagic events by maintaining an FVIII/FIX level>1%.

Nowadays, the FIX used for the treatment of patients suffering from hemophilia B comes from two sources, one being plasma, the other being recombinant (such as Benefix®, Wyeth).

Plasma FIX is prepared from pools of human plasma samples. Viral inactivation and purification methods have significantly reduced the risks of transmission of hepatitis B virus and hepatitis C virus, HIV and other pathogenic agents. A plasma FIX still used today is in particular sold under the name Mononine®, CSL, Behring.

However, in the interests of public health, obtaining recombinant FIX constitutes a safer alternative which is widely preferred because it makes it possible to avoid the problem of the transmission of pathogens that are still unknown and that could be present in the human plasma samples used to prepare plasma FIX.

The cloning and sequencing of the gene encoding FIX between the years 1980 and 1985 made it possible to produce recombinant FIX by means of the cDNA once characterized, and in particular made it possible to identify mutations in most of the patients suffering from hemophilia B (Choo et al., Nature. 1982 Sep. 9; 299(5879):178-80; Kurachi et al., Proc Natl Acad Sci USA. 1982 November; 79(21):6461-4; Yoshitake et al., Biochemistry. 1985 Jul. 2; 24(14):3736-50).

Thus, patients today have the possibility of being treated through the use of recombinant FIX, and in particular with the product sold under the name Benefix®. Benefix® is synthesized by CHO (Chinese Hamster Ovary) cells stably transfected with the FIX cDNA. These cells have a great secretory capacity and are in this respect often used to produce recombinant coagulation factors. However, these CHO cells do not have all the cell machinery necessary for carrying out all the post-translational modifications. Consequently, the Benefix® protein is produced by co-expression of the FIX cDNA with other cDNAs encoding various enzymes necessary for obtaining these post-translational modifications, such as the PACE/furin (Paired basic Amino acid Converting Enzyme) endopeptidase, in order to obtain a functional recombinant protein. However, this co-expression makes it possible to obtain a recombinant FIX of which the gamma-carboxylation is incomplete, and the phosphorylation and sulfation are reduced (Bond et al., 1998. Semin. Hematol. 35, 11-17).

Various processes have been implemented for producing recombinant FIX (with transgenic animals, for example) or for attempting to improve the secretion, the half-life or the activity of FIX, and in particular by improving various post-translational modifications, but these processes have produced results that are more or less satisfactory in terms of production and/or biological activity of the FIX thus produced (Jallat et al., EMBO J. 1990 Oct. 9(10): 3295-301). Scheiflinger et al. (U.S. Pat. No. 7,375,084) have mentioned a factor IX of which the degrees of sulfation and of phosphorylation are increased. White et al. (Transfus. Sci. 19, 177-189, 1998) have studied the pharmacokinetic profiles of plasma FIX (Mononine®) and of recombinant FIX (Benefix®) in patients suffering from hemophilia B. They have demonstrated that the specific activity of the recombinant FIX is equivalent to that of the FIX of plasma origin. On the other hand, the in vivo recovery rate of recombinant FIX is 20 to 30% less than that of plasma FIX, whereas the half-life in the blood stream is similar for each of these two compounds. This difference in recovery rate implies injecting doses of recombinant FIX which are 20 to 30% higher in order to obtain an effective treatment.

Moreover, Chen et al., 1997, Human Gene Therapy 8: 125-135 describe a comparative study on the use of various viral vectors for human FIX expression. Seven retroviral vectors and five of the AAV type were constructed and used to express the human FIX cDNA on a pool of cells of the HepG2 human hepatocyte line and in various fibroblast lines.

Consequently, there is an important need to be able to provide a recombinant FIX which is at least as effective as FIX of plasma origin, and which would ideally exhibit an improved recovery rate in hemophilia B patients.

The present invention makes it possible in particular to solve these problems by providing a novel hepatocyte cell line capable of producing a biologically active recombinant FIX, the activity of which is greater than or equal to that of the recombinant FIX Benefix® in particular.

Thus, according to a first aspect, a subject of the present invention is a recombinant human factor IX (FIX) characterized in that it is obtained by means of a preparation method comprising, or even consisting of, the steps which consist in:
causing the genetic material encoding the FIX to be expressed in vitro in a human hepatocyte cell line Huh7,
recovering the cell supernatant in which the FIX was secreted, and
optionally, purifying the synthesized FIX.

The recombinant FIX of the present invention has in particular the advantage of not requiring any transgene other than the genetic material encoding the FIX. Furthermore, no additional modification of the recombinant FIX thus obtained is necessary in order to obtain a biologically active recombinant FIX.

The term “biologically active FIX” is intended to mean a factor IX of which the specific coagulation activity is at least 100%. The recombinant FIX secreted into the culture medium of the present invention preferably has a specific coagulation activity of from 150 to 200%, preferably from 170 to 200%. The specific coagulation activity of the factor IX is determined by means of the technique termed chronometric time technique by measuring the partial thromboplastin time (PTT) or activated partial thromboplastin time (aPTT) which is a semi-overall test of blood coagulation which uses a coagulometer. The test used is detailed in the examples. In this test, Benefix® has a specific activity of 100%.

Preferably, the Huh7 hepatocyte cell line used is the line deposited at the ATCC under number CCL-185.

According to one particularly advantageous embodiment, the recombinant FIX of the present invention is obtained by using the genetic material encoding FIX only, without any other genetic material(s) encoding other proteins.

Preferably, no structural modification is introduced into the recombinant FIX protein obtained. Indeed, the recombinant FIX protein obtained has already undergone post-translational modifications through the intrinsic system of the hepatocyte cells, which is sufficient in many cases.

According to one preferred embodiment of the present invention, the genetic material encoding the FIX which is used is a cDNA. Preferably, the cDNA used is the full length cDNA of wild-type human factor IX (Genbank No. NM_{—}000133) comprising the polymorphism Thr148Ala, nucleotide A20422G (McGraw et al., Proc Natl Acad Sci USA 1985 May; 82(9): 2847-51). It is also possible to use the cDNA of human factor FIX comprising the truncated intron 1, the construction being obtained according to Kurachi et al. (J Biol Chem. 1995 Mar. 10; 270(10): 5276-81) and developed by Enjolras N. et al. (Thromb Haemost. 1999 October; 82(4): 1264-9).

Any method for transferring genes into and expressing them in eukaryotic cells that are well known to those skilled in the art can be used to prepare the recombinant FIX according to the invention. Among these methods, mention may in particular be made of transfection by lipofection, or transfection by calcium phosphate precipitation.

Use may preferably be made of transfection, in particular by lipofection, and in particular stable transfection so as to have a constant and stable production of recombinant protein.

The genetic material encoding the FIX is expressed by means of any type of expression vector or system. Preferably, the vector used is nonviral.

Once the FIXwt cDNA has been transferred, the cells are maintained under culture conditions suitable for the type of cells used and which allow the expression and the secretion of the recombinant FIX (presence of vitamin K1 in the culture medium), or at the very least conditions which are not such that they prevent the expression, maturation and secretion of the FIX.

Advantageously, the recombinant human factor IX (FIX) according to the invention has an electrophoretic profile identical to the plasma form of human FIX, in particular to Mononine®.

Furthermore, the recombinant FIX of the invention has the post-translational modifications necessary for obtaining satisfactory coagulation activity. In particular, the recombinant FIX of the invention is N-glycosylated and/or sialylated.

Moreover, the activation peptide of the recombinant FIX according to the invention, i.e. amino acids 192 to 226 of SwissProt reference P00740, is present in a phosphorylated and/or sulfated form, and preferably phosphorylated and sulfated form.

According to a second aspect, a subject of the present invention is a method for preparing a human hepatocyte cell line producing biologically active recombinant human FIX, characterized in that it comprises, or even consists of, the following steps:
causing the genetic material encoding the FIX to be expressed in vitro in said human hepatocyte cell line Huh7,
recovering the cell supernatant in which the FIX was secreted, and
optionally, purifying the synthesized FIX.

Preferably, a purification of the FIX produced by the hepatocyte line is carried out. The purification of the synthesized FIXwt can be carried out using the culture medium of the producer clone obtained.

All the preferred embodiments which are stated above as regards the recombinant FIX of the invention are also applicable without restriction to the method.

According to a third aspect, a subject of the present invention is the recombinant FIX which is produced by the Huh7-CD4 cell line that was deposited on Oct. 20, 2009, with the Collection Nationale de Cultures de Microorganismes [National Collection of Microorganism Cultures] of the Pasteur Institute, 25 rue du Docteur Roux in Paris (France) and registered under number I-4234.

According to a fourth aspect, a subject of the present invention is also the I-4234 cell line as such.

The present invention is described in greater detail in the nonlimiting examples which follow and which refer to the appended figures in which:

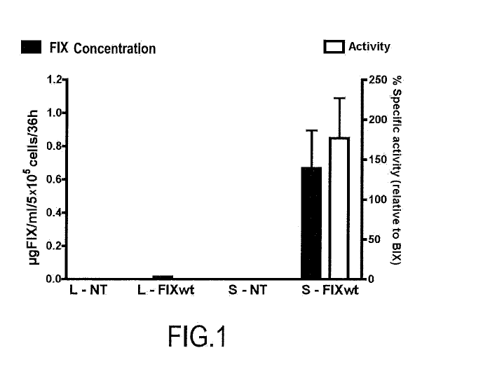

FIG. 1 represents the FIX concentrations (▪) (in μg FIX/ml/5×10⁵ cells) measured in the cell lysates of Huh7 cells transfected with pcDNAFIXwt (L-FIXwt) or nontransfected Huh7 cells (L-NT), after 36 hours, and the specific activity of the FIX (□) measured in the supernatants of Huh7 cells transfected with pcDNAFIXwt (S-FIXwt) or of nontransfected Huh7 cells (S-NT), 36 h after transient transfection.

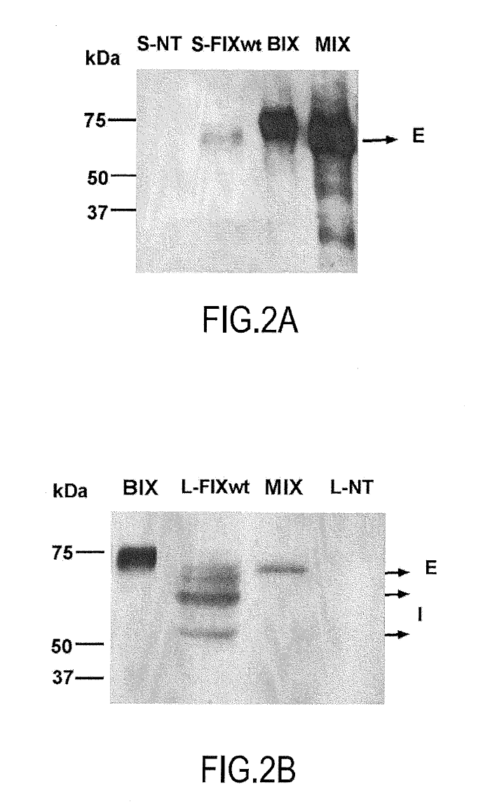

FIG. 2 represents the Western blot obtained for the supernatants (A) and the total lysates (B) of Huh7 cells transfected with pcDNAFIXwt (FIXwt) or of nontransfected Huh7 cells (NT). Benefix® (BIX) and Mononine® (MIX) are used as controls. A Biorad standard molecular weight (MW) marker was used as a reference.

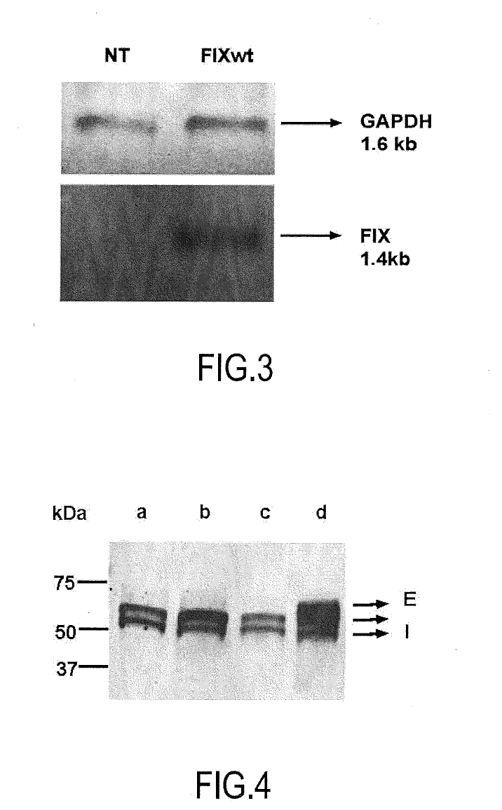

FIG. 3 represents a Northern blot using the total RNAs of Huh7 cells transfected with pcDNAFIXwt (FIXwt) or of nontransfected Huh7 cells (NT) after hybridization overnight with a probe corresponding to the whole cDNA of human FIX—or a probe corresponding to the cDNA of rat GAPDH as a control, and 30 minutes of exposure.

FIG. 4 represents a Western blot obtained from lysates of Huh7 cells transiently transfected with pcDNAFIXwt, and treated with protein degradation inhibitors NH₄Cl (b), clasto-lactacystin (β-lactone) (c) and brefeldin A (d), or without treatment (control (a)).

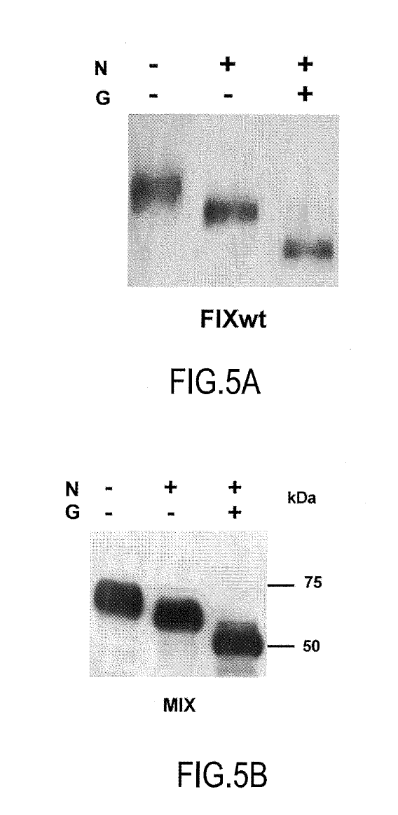

FIG. 5 (A) represents the Western blot obtained from supernatants of transfected Huh7 cells (FIXwt). These supernatants were treated (+) or not treated (−) with neuraminidase (N) or N-glycosidase (G). FIG. 5 (B) represents the Western blot obtained from Mononine® (MIX) treated (+) or not treated (−) with neuraminidase (N) or treated (+) or not treated (−) with N-glycosidase (G).

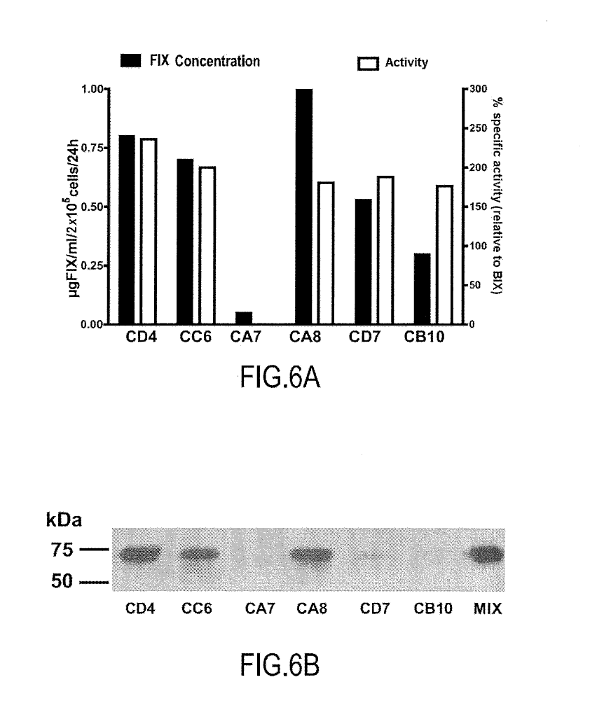

FIG. 6 represents:

(A) the FIX concentrations (▪) and the corresponding FIX specific activity (□) measured for each clone obtained from Huh7 cells stably transfected with pcDNAFIXwtI1.

(B) The Western blot obtained from the supernatants of the 6 clones selected after stable transfection of Huh7 cells with pcDNAFIXwtI1.

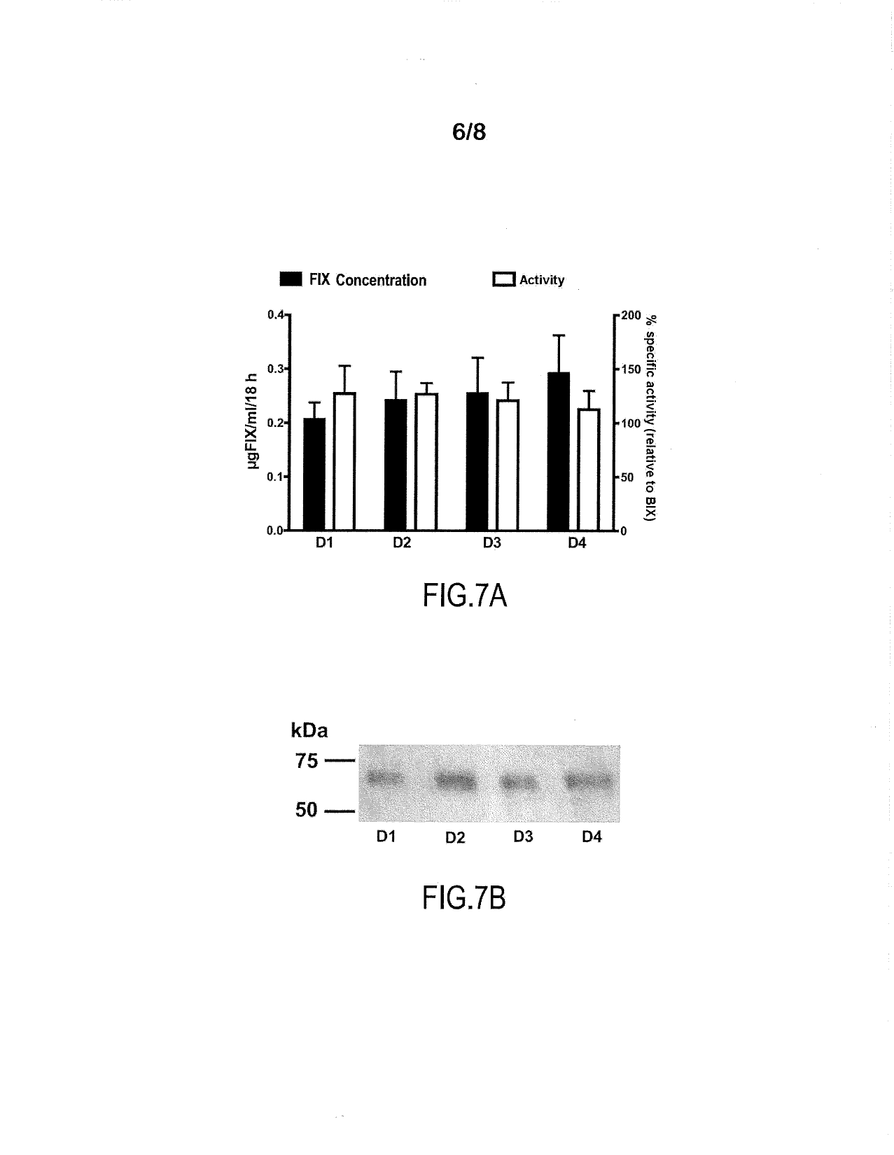

FIG. 7 represents:

(A) the FIX concentrations (▪) and the FIX specific activity (□) measured for each sample collected each day for 4 days (D1 to D4 respectively) from the culture of the Huh7-CD4 clone in roller bottles.

(B) The Western blot which makes it possible to visualize the FIX obtained from the supernatants of the samples collected each day (D1 to D4) for 4 days.

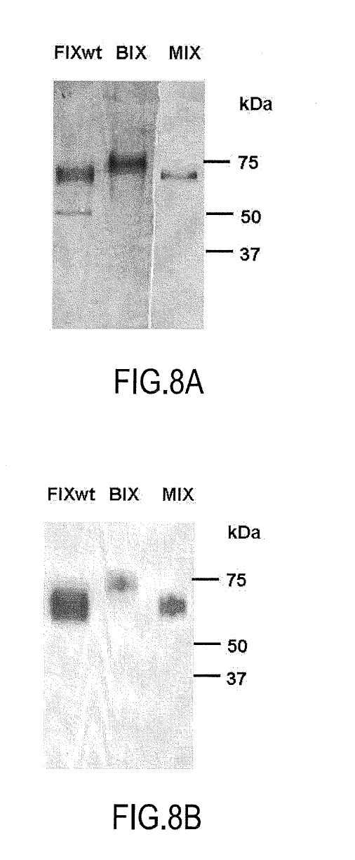

FIG. 8 represents:

(A) an electrophoresis by SDS-PAGE of the purification of factor IX (FIXwt) (100 ng) from the culture supernatants of the Huh7-CD4 clone. Mononine® (MIX) and Benefix® (BIX) were loaded as controls. The proteins are visualized after silver nitrate staining.

(B) Electrophoresis of the factor IX (FIXwt) (100 ng) purified from the culture supernatants of the Huh7-CD4 clone. Mononine® (MIX) and Benefix® (BIX) were loaded as controls. The proteins were visualized after immunodetection using a polyclonal antibody.

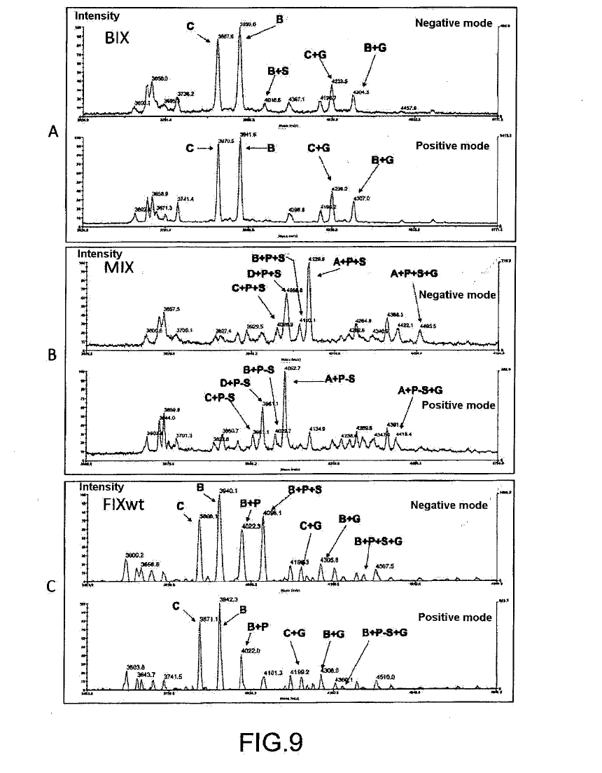

FIG. 9 represents the MALDI-TOF mass spectra after treatment on ZrO₂ demonstrating the post-translational modifications such as phosphorylation and sulfation of the activation peptides present in samples of FIX according to the invention, of BIX and of MIX.

### EXAMPLES

Obtaining the Expression Vector

The 1.4 Kb full-length cDNA of human FIX was cloned into pcDNA3.1 (Invitrogen, Cergy Pontoise, France) in order to obtain the plasmid pcDNAFIXwt, an expression vector with the cytomegalovirus (CMV) promoter and the human FIXwt cDNA (Enjolras et al., 1999, above, according to Kurachi et al., 1995, above).

Cell Culture and Transient Transfection

The Huh7 human hepatocyte carcinoma cell line (ATCC No. CCL-185) was maintained in HYQ medium (Thermo Scientific, Logan, Utah), supplemented with 10% of fetal calf serum (FBS) (Perboscience, Brebières, France), 1% of PS (pencillin, streptomycin), 1% of L-glutamine and 5 μg/ml of vitamin K1 (Roche, Neuilly-sur-Seine, France) at 37° C. in a humid atmosphere containing 5% CO₂. Twenty hours before transfection, the Huh7 cells were plated out in 6-well plates at a density of 5×10⁵ cells/well. The Huh7 cells were transfected with 1 μg per well of pcDNAFIXwt recombinant vector using FuGENE-6° (Roche, Neilly-sur-Seine, France) according to the supplier's instructions. Twenty-four hours after transfection, the cells are incubated by adding fresh, serum-containing medium.

Culture Media, Cell Lysates and Western Blotting

The Huh7 cells (5×10⁵/well) were or were not transfected with pcDNAFIXwt in 6-well plates. Thirty-six hours after transfection, the supernatants were collected. The cells were then washed with PBS and incubated for 16 h in serum-free medium. The serum-free supernatants were harvested and the cell extracts were prepared in 300 μl (for 2×10⁶ cells) of cold 0.5% Triton 100× lysis buffer (100 mM/l KCl, 2 mM/l MgCl₂, 10 mM/l Hepes, pH 7.5, 0.5% Triton-X100) containing a complete mixture of antiproteases (Complete® antiprotease mix (Roche, Mannheim, Germany)). An aliquot of the resulting solution (10 μl) (serum-free supernatants or cell lysastes) was subjected to electrophoresis on a denaturing 10% polyacrylamide gel (SDS-10% PAGE). The gels were blotted onto Hybond C® Pure membranes (GE Healthcare, Orsay, France). The membranes were blocked with TBS-T-milk (0.15 mM/l NaCl, 10 mM/l Tris-HCl, pH 7.5, 0.1% Tween-20, powdered milk) overnight at ambient temperature, and then incubated for 1 h with a rabbit anti-human FIX polyclonal antibody diluted to 1:200 in TBS-T-milk (Régilait). The membrane was then washed 3 times in TBS-T and then incubated for 30 min with a peroxidase-labeled anti-rabbit antibody diluted to 1:3000 (Biorad, Ivry sur Seine, France) in TBS-T-milk. After 3 washes, the chemiluminescence was measured by autoradiography using the System system (GE Healthcare).

ELISA Assay and Specific Coagulation Activity

The FIX concentrations in the culture medium and the cell extracts were measured 36 hours after transient transfection using the Asserachrom IX:Ag ELISA kit (Diagnostica Stago).

The specific coagulation activity of the factor IX is determined using the technique termed chronometric time technique by measuring the partial thromboplastin time (PTT) or activated partial thromboplastin time (aPTT) according to K. J. Smith et al., Blood, 72, 1269-1277 (1988), which is a semi-overall one-step blood coagulation test with an MDA II® or else Destiny Max coagulometer (Trinity Biotech, Dublin, Ireland). For this, fifty microliters of sample are diluted to 1/10th in imidazole buffer (Trinity Biotech, Bray, Ireland). Fifty microliters of diluted sample are added to 50 of factor IX-deficient plasma (Precision Biologic, Darmouth, Canada) and 50 μl of MDA Platelin (Kordia, Leiden, the Netherlands). After 3 minutes of incubation, the coagulation is initiated by adding 50 μl of MDA Platelin LS® 25 mM CaCl₂ (25 mM). The coagulation time is measured by means of a coagulometer using an MDA II® or else Destiny Max automated device (Trinity Biotech, Bray, Ireland). The activity of the factor IX is determined from standard plasma values (Standard Human Plasma, Siemens Marburg, Germany, calibrated against a WHO—World Health Organization—standard) by means of a log-log curve. The values were then related back to those of Benefix® which has a specific activity of 100%, in the knowledge that 5 μg/ml of FIX has an activity of 100%. Thus, the specific activity of the FIXwt of the samples was calculated according to the ratio between the coagulant activity of the sample related back to the values of those of Benefix® and the FIXwt concentration of this same sample (aPTT/ELISA).

A comparative study with respect to the works of Chen et al., 1997, was carried out. In these experiments, 1×10⁶ cells of the Huh7-CD4 clone were seeded into 6-well plates. The following day, the medium was renewed and left in the presence of the producer cells for 24 hours. The FIX concentrations in this medium are determined using the Asserachrom IX:Ag ELISA kit (Diagnostica Stago). The activity of the FIX secreted into this medium is determined in a one-step coagulation test (aPTT) with an MDA II or else Destiny Max coagulometer (Trinity Biotech, Dublin, Ireland) as described above. The activity of the factor IX is determined from standard plasma values (Standard Human Plasma, Siemens Marburg, Germany, calibrated against a WHO—World Health Organization—standard) by means of a log-log curve. In order to allow comparison of the values of the FIXwt of the invention with those of the FIX synthesized by HepG2 cells, cited in the works by Chen et al., 1997, the coagulant activities were not therefore related back to those of Benefix® in this case, but to that of the internal standard of the coagulometer. The specific activity of the FIX resulting from the Huh7-CD4 clone is calculated according to the ratio between the coagulant activity read for the sample, related back to the standard plasma values, and its FIX concentration (aPTT/ELISA). In order to obtain units of activity comparable to those of Chen et al., the aPTT values for the FIXwt of the invention are converted into ng/ml, in the knowledge that 5 μg/ml of FIX has an activity of 100%.

Northern Blot

The total RNAs were prepared, 36 hours after transfection, from 5×10⁵ Huh7 cells transiently transfected with pcDNAFIXwt or not transfected, using the Rneasy Mini Kit (Qiagen, Courtaboeuf, France).

After electrophoresis on 0.8% agarose gel in a phosphate buffer (10 mM/l NaH₂PO₄, 10 mM/l Na₂HPO₄, pH 7) using 500 ng of total RNAs, the mRNAs were blotted onto a Hybond N® nylon membrane (GE Healthcare).

The plasmids containing the full-length cDNA of human FIX and that of rat GAPDH are those described by Enjolras et al., 1999, above. The RNA probes containing the antisense sequence of human FIX and of rat GAPDH were generated and labeled with NTPs containing UTPs-digoxigenin (DIG), using the in vitro transcription system of the T7 RNA polymerase kit (Roche) and according to the protocol described by Enjolras et al., 1999, above. The prehybridization, hybridization and washing steps were carried out according to Roche's recommendations.

The membranes were incubated overnight with the two types of RNA probes. The signals were detected after 4 hours with the DIG Luminescent Detection Kit (Roche).

Effects of Protein Degradation Inhibitors on the Intracellular Level of FIXwt

Huh7 cells (1×10⁶ cells/dish) were transiently transfected at 80% confluence in 60 mm culture dishes. Thirty-six hours after transfection, the cells were incubated in FBS-free medium containing 10 μM/l of clasto-lactacystin β-lactone (Calbiochem, France Biochem, Meudon, France), 50 mM/l of ammonium chloride, or 10 μg/ml of brefeldin A (Sigma Aldrich). The medium was renewed every 2 hours.

The cell lysates were harvested 6 h later and prepared in 120 μl of cold lysis buffer. The FIX antigen concentration was quantified on the lysates by means of an ELISA assay. The results are expressed as percentage of values obtained relative to the nontreated control lysates. The comparisons were carried out by application of Fisher's test using the Stat View® software.

Analysis of Sialylation and N-Glycosylation

The supernatants of transiently transfected Huh7 cells were collected 36 hours after transfection. The cells were washed with PBS and incubated for 16 h in serum-free medium. These media were harvested and they were incubated with 100 mU/ml of neuraminidase (Roche) for 1 h at 37° C., and with 9.4 U/ml of peptide-N4-(N-acetyl-β-glycosaminyl)asparagine amidase (recombinant N-glycosidase F) (Roche) for 16 h at 37° C. in 20 mM/l of Tris-maleate, pH 6.0, 150 mM/l NaCl, 5 mM/l calcium chloride and 1.75% (vol/vol) of NP-40. The digestions were stopped by adding 2× Laemmli buffer containing SDS and β-mercaptoethanol, and the digestion products were subjected to SDS-10% PAGE electrophoresis under denaturing conditions. The FIX was then visualized by immunoblotting as described above. The same protocol was applied for 100 ng of Mononine® (MIX).

Stable Transfection and Selection of the FIX-Producing Huh7 Clones

Huh7 cells were seeded into petri dishes (1×10⁶ cell/dish). The Huh7 cells were transfected with 2 μg of recombinant pcDNAFIXwtI1 vector using FuGENE-6® (Roche, Neuilly-sur-Seine, France) according to the supplier's instructions. This plasmid contains the truncated intron 1 of the human FIX gene, and was previously shown to have an activity which increases expression in HepG2 cells (Kurachi et al., 1995, above) and in CHO cells (Enjolras et al., 1999, above). This pcDNAFIXwtI1 plasmid contains the geneticin (G418) resistance gene. Twenty-four hours after transfection, the cells were brought into contact with medium containing geneticin (G418) (350 μg/ml). The G418-resistant clones were obtained 3 weeks after transfection. The clones were subcultured individually (without trypsin) and re-seeded into 96-well plates containing 200 μl of culture medium. The amount of FIX was evaluated in each supernatant from cells at confluence, and the clones producing the most FIX were amplified in 24-well plates. During the passaging of the FIX-expressing clones from 96-well plates to 24-well plates and from the 24-well plates to 12-well plates and from the 12-well plates to 6-well plates, the cells were detached mechanically using a scraper or a tip and were therefore never trypsinized. This method of passaging during the amplification of the cells made it possible to successfully obtain the clones described in this text. When the cells showed 80% confluence in a 75 cm² flask, they were frozen in liquid nitrogen for storage in complete medium containing 90% FBS and 10% DMSO.

The producer clone was chosen during the passaging of the cells into a 24-well plate. The supernatants of six clones which were the highest FIX producers were seeded in a proportion of 2×10⁵ cells per well. The following day, the culture medium was replaced with complete medium containing 10% FBS in the presence of geneticin. After 24 hours of incubation, the culture medium of each clone was harvested and the FIX concentration and also the specific activity were measured. The cells were rinsed and incubated for the next 18 hours in serum-free medium. After this incubation, the media were harvested. 10 μl aliquots were loaded onto an electrophoresis gel. The FIXs secreted by the various clones were visualized by Western blot. This step made it possible to choose the Huh7 clone intended for FIX production.

Production of Recombinant FIX Derived from Supernatant of the Huh7-CD4 Line

The cells of the Huh7-CD4 clone were amplified at a rate of two 1-in-3 passages per week. During the two weeks of amplification, the geneticin concentration was gradually increased to a value of 0.4 mg/ml. The cells were seeded into roller bottles, in a proportion of 30×10⁶ cells per roller bottle. These roller bottles have a capacity of 1700 cm² (Cellmaster rollerbottles, Ref 682065, Greiner Bioone, Dutscher) and are used with an IBS CellRoll rotating system (Integra Biosciences). Seventy-two hours later, the cells were washed and brought into contact with serum-free medium, and then incubated for 18 hours. The medium was then removed and freed of the cells in suspension and other cell debris by centrifugation (3000 rpm, 20 min). The cells were brought into contact with complete medium for 12 hours. This production protocol was repeated three times for the same roller bottle, the cells having been used for the production were not preserved. The media containing the FIXwt were preserved by freezing at −30° C.

An aliquot of each serum-free culture supernatant, taken every day of production, was subjected to FIX quantification using an FIX ELISA kit, and then to a coagulation measurement by means of the chronometric time method on an MDA II or else Destiny Max automated device. The measurements were carried out after a first thawing of the media.

Aliquots of these culture supernatants thus harvested (10 μl) were subjected directly to polyacrylamide gel electrophoresis (SDS-PAGE/10%). The FIX was visualized in each supernatant by Western blot.

Purification of Recombinant Fix Derived from Supernatants of the Huh7-CD4 Clone

An ion exchange column (High Trap Q FF, 5 ml, GE Healthcare Ref: 17-5156-01) was used during the first purification step. The column was pre-equilibrated with 50 mM Tris buffer, pH 8. It was loaded with a volume of from 250 to 300 ml of Huh7-CD4 cell supernatant containing, on average, 0.2 μg/ml of FIX, and filtered on a 0.2 μm filtration unit. The column was then washed with a 50 mM Tris buffer, pH 8. The FIX was eluted with 50 mM Tris buffer, pH 8, containing 1M NaCl. The fraction containing the FIX (approximately 30 ml) is collected.

A second purification step consisted of chromatography using an immunoaffinity column. The immunoaffinity column was produced by covalently coupling a monoclonal antibody directed against FIX (Centeon, Kankakee, Ill., USA) to Dynabeads MyOne Tosylactivated magnetic beads (Invitrogen Ref 655.01). The bead/antibody coupling protocol was followed according to the supplier's instructions.

The immunoaffinity column was pre-equilibrated with PBS-0.05% Tween buffer, pH 7.4. The beads (500 μl) were brought into contact with 10 ml of eluate containing the FIX resulting from the ion exchange column (2 μg/ml of FIX). The incubation was carried out overnight at 4° C. with rotary shaking. The column was then washed twice with PBS-0.05% Tween. The FIX was eluted with 500 μl of 0.5 M glycine elution buffer, pH 2.1. The eluate was loaded onto a PD10 desalting column (GE Healthcare). The FIX is eluted with 1 ml of 50 mM Tris-HCl buffer, pH 8, containing 0.1 M NaCl, and concentrated on a concentrating system of which the cut-off capacity is at 30 kDa. The quality of the eluate was verified by loading 100 ng onto an electrophoresis gel. The visualization was carried out by a silver nitrate staining and by Western blot.

Mass Spectrometry Analysis of the FIX Sulfation and Phosphorylation

The sulfated and phosphorylated peptides of the FIXs were detected by MALDI-ToF in negative mode.

Ten micrograms of purified FIXwt, of MIX and of BIX were deglycosylated using a deglycosylation kit (QAbio-Euromedex, Souffelweyersheim, France) containing a mixture of sialidases and of O- and N-glycosidases, for 16 h at 37° C. The digestions were stopped by adding 2×Laemmli buffer containing SDS and β-mercaptoethanol. The digestion products were subjected to SDS-10% PAGE electrophoresis under denaturing conditions. The deglycosylated FIX bands were stained with Coomassie blue so as to be cut out from the gel. After reduction with 10 mM DTT and alkylation with 100 mM iodoacetamide, they were subjected to digestion with sequence grade trypsin (Promega, Charbonnières-les-Bains, France). The tryptic peptides were recovered and placed in acid solution so as to then be analyzed by LC/MS/MS (LC: Ultimate 3000 Dionex, MS LTQ Velos ThermoScientific, ionization nanospray). The column is a Dionex PepMap C18 nanocolumn (75 μm×15 cm) adjusted to a flow rate of 300 nl/min.

The same tryptic solutions were pretreated on a zirconium oxide Tip (Supel-Tips Zr, pipette tips of volume 10 μl, Supelco 54266-U, supplier's protocol). This phase made it possible to preferentially retain the phosphorylated and/or sulfated peptides. The extracts were analyzed on a Voyager DE-PRO apparatus (AB Sciex), in negative linear mode for a matrix of 2′,4′,6′-trihydroxyacetophenone with added ammonium citrate. Only the average masses (=chemical masses) were considered.

By MALDI-TOF, the sulfated peptides (sulfated on tyrosine) could be detected in the negative mode in [M−H]⁻ form. In the positive mode, only the desulfated ion [M+H—SO₃]⁺, i.e. [M+H-80]⁺, was observed. The peptides phosphorylated on serine/threonine have, for their part, a different behavior: the [M−H′]⁻ ions in the negative mode and [M+H]⁺ ions in the positive mode, without loss of PO₃ or H₃PO₄.

### Results

### Example 1

### Expression, Biosynthesis and Activity of the FIXwt Produced by the Transiently Transfected Huh7 Cells

Huh7 cells were transiently transfected with the pcDNAFIXwt plasmid containing the wild-type FIX cDNA (FIXwt) under the control of the CMV promoter. After 36 h of transfection, the FIX concentrations in the total cell lysates and culture supernatants were measured using the ELISA kit and the specific activity in the culture supernatants was measured using a one-step coagulation test. The results of the values representative of three transfections are brought together in the appended FIG. 1.

These results show that the Huh7 cells thus transfected have the ability to secrete between 0.5 and 0.6 μg/ml/36 h/5×10⁵ cells of FIX (S-FIXwt). The intracellular FIX levels (L-FIXwt) reach 0.05 μg/ml of cell lysate. No FIX was detected in the lysates (L-NT) and supernatants derived from the nontransfected Huh7 cells (S-NT).

The specific activity measured on the supernatants of the transfected cells is between 150 and 200% relative to that of Benefix®, which is 100%.

The lysates and the supernatants were subjected to electrophoresis, then analyzed by Western blot in order to reveal the FIX produced. The results are brought together in the appended FIG. 2.

The results concerning the supernatants (FIG. 2(A)) show that the factor IX (S-FIXwt) secreted is visualized in the form of a single band (band E) with an apparent molecular weight of 65 kDa and has the same mobility as Mononine®, whereas Benefix® (BIX) migrates slightly more slowly. No FIX signal was detected in the medium (S-NT) derived from the nontransfected Huh7 cells.

The results concerning the lysates (FIG. 2(B)) show three major bands for the transfected Huh7 cells (L-FIXwt). The highest band (band E) has the same molecular weight as that of the secreted form or as that of Mononine (MIX), Benefix® (BIX) still migrating more slowly. The two other bands (bands I) migrate slightly faster (57 kDa) than band E. These results show that the Huh7 cells expressing the FIX contain two forms of intracellular FIX, one corresponding to a form which can be secreted (band E) and the other corresponding to a doublet (bands I) which corresponds to various degrees of post-translational modifications.

In order to study the transcription efficiency for the pcDNAFIXwt plasmid, the total RNAs were extracted from the transfected or nontransfected Huh7 cells 36 h after transfection. The RNAs were analyzed by Northern blot with a “full-length” FIX RNA probe. The results are brought together in the appended FIG. 3.

A single FIX transcript was detected in the transfected cells (FIXwt), whereas the nontransfected Huh7 cells (NT) contain no detectable endogenous transcript. The mRNAs are present in similar amounts in the two types of samples, as indicated by the signal obtained for the GAPDH reference.

### Example 2

### Effects of Protein Degradation Inhibitors on the Biosynthesis of Intracellular FIX

The results above (FIG. 1) show that the FIXwt is present at a low intracellular level in the transfected Huh7 cells, which means that these cells do not accumulate the FIX.

The effect of various protein degradation and cell trafficking inhibitors was tested on the Huh7 cells transfected with pcDNAFIXwt.

Thirty-six hours after transfection, the inhibitors were added to the culture medium and left for 6 h. The intracellular FIX concentrations were measured after treatment by means of an ELISA assay (the values are expressed as % of FIX relative to the values obtained in the nontreated cell lysates). The results are brought together in table 1 below and correspond to three independent transfections.

TABLE 1

| | Intracellular FIX level |
|---|---|
| Treatments | (mean ± SE) |
| Control (without treatment) | 100 |
| NH₄Cl (50 mM/l) | 84.7% +/− 28.1 |
| Beta-lactone (10 μM/l) | 50.1% +/− 25.8 |
| Brefeldin A (10 μg/ml) | 254.4% +/− 21.4 |

The proteasome-specific inhibitor clasto-lactacystin β-lactone has no significant effect on the intracellular FIXwt level. Likewise, NH₄Cl, which inactivates lysosomal enzymes by modifying the pH, does not significantly increase the FIXwt level (50.1% with NH₄Cl compared with 100% without NH₄Cl). Brefeldin A blocks protein transport from the endoplasmic reticulum (ER) to the Golgi complex and causes return translocation of components from the Golgi to the ER. After incubation with brefeldin A, the intracellular FIXwt level increases by 370% compared with that detected without brefeldin A (p=0.005).

The lysates of the treated cells were also subjected to electrophoresis, and the FIXwt was detected by Western blot as indicated above. The results are brought together in the appended FIG. 4 and correspond to six independent transfections.

The FIX of the nontreated cells exhibits the same profile as that previously observed (FIG. 2), the secreted form (band E) and the doublet (bands I), lane a. When the cells are treated in the presence of NH₄Cl (lane b) or of β-lactone (lane c), the intensity of the bands decreases according to the FIX ELISA values; the E form does not vary in intensity.

Conversely, in the presence of brefeldin A (lane d), all the intracellular FIX forms accumulated in the cells and in particular the secreted E form.

These results show that the intracellular FIXwt forms are barely or not at all degraded in the transfected Huh7 cells and that they cross the ER barrier in an efficient manner so as to be rapidly secreted. The recombinant FIX synthesized by the Huh7 cells according to the invention therefore takes a conventional intracellular maturation pathway.

### Example 3

### Analysis of Post-Translational Modifications of the Secreted FIXwt

In order to study the N-glycosylation and the sialylation of the FIXwt secreted by the transiently transfected Huh7 cells, the culture supernatants were incubated with neuraminidase and an N-glycosidase.

The digestion products were subjected to reducing electrophoresis and analyzed by Western blot as described above. The results are brought together in the appended FIG. 5.

These results show that the secreted form of the FIXwt (FIG. 5(A)), left-hand panel, is found with a molecular weight of 65 kDa as described above. A slight decrease in the molecular weight of the FIXwt is observed following digestion with neuraminidase (N+, G−) and an additional reduction is also observed after N-deglycosylation (N+, G+).

The same migration profiles were observed following the treatment of Mononine® (plasma FIX of the prior art) under the same digestion conditions (right-hand panel (FIG. 5(B))).

These results show that the FIXwt secreted by the Huh7 cells transiently transfected with pcDNAFIXwt is N-glycosylated and sialylated in the same way as is Mononine®, which corresponds to a mature secreted FIX. The set of results described in examples 1 to 3 above demonstrates that the Huh7 cells transiently transfected with pcDNAFIXwt are capable of producing a biologically active recombinant FIX which has a specific activity improved by a factor of 1.7. The protein produced is correctly synthesized, and follows the conventional intracellular trafficking pathways before being secreted. The N-glycosylation and sialylation are correctly preformed, and the FIXwt is not retained in the cells. These data demonstrate that these Huh7 cells represent an effective cell system for producing a biologically active recombinant FIX.

### Example 4

### Obtaining an Huh7 Cell Line which Stably Secretes Recombinant FIX

Huh7 cells were stably transfected with the pcDNAFIXwtI1 plasmid. Each G418-resistant clone was subcultured individually so as to be amplified as indicated above.

Six clones which produce FIX at concentrations above 0.1 μg/ml were selected. They were seeded into 24-well plates in a proportion of 2×10⁵ cells per well, and maintained under 5% CO₂ in HyQ culture medium supplemented with fetal calf serum (10%) for 24 h. The cells were then washed twice with PBS and 500 μl of serum-free medium was added, and the supernatants were collected after 24 h of incubation.

The production and the specific activity of the FIX present in the medium of each of these 6 clones were measured. Aliquots derived from the serum-free supernatant of these 6 clones were then subjected to electrophoresis and the signals were detected by autoradiography. The results are brought together in the appended FIG. 6.

These results (FIG. 6(A)) show that three of the six clones (CD4, CC6 and CA8) exhibit an FIX production of up to 0.75 μg/ml with a total FIX activity, and two clones (CD4 and CA8) exhibit FIX concentrations of between 0.75 and 1 μg/ml with a total FIX activity. Similar improved specific activities were found here as previously shown for the transiently transfected Huh7 cells.

Furthermore, the Western blot results (FIG. 7(B)) show that each producer clone exhibits a single FIX band at the expected molecular weight.

The CD4 clone exhibits the best concentration/specific activity combination, which makes it a clone of choice for producing recombinant FIX. The Huh7-CD4 cell line derived from the CD4 clone and which represents one of the aspects of the invention could be subcultured by means of two passages per week with a 1:3 subculturing ratio.

### Example 5

### Production of FIX in Roller Bottles by the Huh7-CD4 Cell Line in Serum-Free Medium

Roller bottles of 1700 cm² (Cellmaster rollerbottles, Ref 682065, Greiner Bioone, Dutscher) were used with an IBS CellRoll rotary system (Integra Biosciences). Three million cells of the Huh7-CD4 clone were maintained in HyQ medium supplemented with 10% FBS for 72 h in roller bottles at 37° C. under 5% CO₂.

On day 1, the cells were washed twice with PBS buffer and serum-free medium (25 ml per roller bottle) was added for 15-18 hours. The supernatants were collected and represent the first sample of FIX production (day 1 or D1). The cells were then maintained for 10 h in complete medium and the FIX production in serum-free medium was repeated for a further 3 days, while alternating with the steps of incubation in complete medium.

The FIX production in the 4 samples collected from day 1 to day 4 (D1 to D4, respectively) was measured by means of an ELISA assay, and the specific coagulation activity of each sample was measured as indicated above. The presence of FIX was also verified by Western blot. The results are brought together in the appended FIG. 7.

The results of FIG. 7(A), expressed as mean value +/−SD from 4 productions for 4 days, show that the FIX concentrations are 0.2 μg/ml on day 1 (D1) to day 4 (D4), which is the final day of the production period. Furthermore, a total specific activity is preserved over the course of the four days of culture.

The results of FIG. 7(B) also show that the FIX is detected in each sample D1 to D4, which makes it possible to conclude that the FIX clearly continues to be secreted with the same quality over the course of the period of production in roller bottles.

### Example 6

### Characterization of the Purified FIX by Western Blot and Measurement of Specific Activity

The recombinant FIX produced by the Huh7 line was purified by means of an ion exchange chromatography step and an immunoaffinity step as described in the results. An aliquot of 100 ng of recombinant FIX, and 100 ng of Mononine® and of Benefix® was subjected to electrophoresis. The quality of the FIXs was visualized by protein staining with silver nitrate (FIG. 8(A)) and by Western blot (FIG. 8(B)). As shown in FIG. 8(A), the purified recombinant FIX (FIXwt) exhibits a band at 65 kDa, having the same mobility as the signal corresponding to Mononine® and a slightly faster mobility than that of Benefix®. A band at 50 kDa is visible in the sample. It corresponds to a very weak contamination with anti-FIX antibody from the immunoaffinity column. Indeed, a fraction of the antibody is co-eluted with the FIX.

These observations are confirmed by the Western blot experiments (FIG. 8(B)). The purified FIXwt exhibits a mobility identical to that of Mononine® and slightly faster than that of Benefix®.

### Example 7

### Comparison Example

Chen et al., 1997, Human Gene Therapy 8: 125-135 used viral vectors to produce FIX, in particular in the HepG2 hepatocyte cell line.

The results of the ELISA and aPTT assays obtained that were published by these authors are reproduced in the first three lines of table 2 below.

Moreover, the specific activity of the FIX produced by the Huh7-CD4 line was determined according to the conditions determined by Chen et al. The ratio between the concentrations measured by the aPTT method (ng/ml) and measured by ELISA (ng/ml) was calculated on the basis of five different assays. The concentration values were determined in ng of FIX/24 hours/10⁶ cells according to the conditions determined by Chen et al.

TABLE 2

| | ELISA | aPTT | % specific activity |
|---|---|---|---|
| Molecules | (ng/ml) | (ng/ml) | aPTT/ELISA |
| FIX/GCF9SN-MPSV | 76 | 55 +/− 15 | 73.5 +/− 19.7 |
| FIX/GCNCF9 | 128 | 120 +/− 14 | 93.7 +/− 11 |
| FIXwt/Huh7-CD4 | 461 +/− 12 | 740 +/− 59 | 161 +/− 37 |

These results demonstrate that the FIX according to the invention has an improved specific activity compared with that of the recombinant FIX produced according to the method described by Chen et al.

### Example 8

### Analysis of the Sulfation and Phosphorylation of the FIXwt According to the Invention

The detection of the sulfated and phosphorylated peptides of the FIXs was carried out as described above.

The multicharged-molecule spectra obtained are brought together in FIG. 9.

The peptide of interest corresponding to the FIX activation peptide [amino acid 192-amino acid 226] (according to SwissProt reference P00740) was studied in this assay and it was found in various forms. The following distinction was used for the various forms of activation peptide that were identified:
form A: wild-type peptide [192-226] according to SwissProt: P00740=(₁₉₂AETVFPDVD . . . FTR₂₂₆) theoretical mass 3969.2,
form B: variant peptide [192-226] T/A at 194=(₁₉₂AEAVFPDVD . . . ₂₂₆), theoretical mass 3939.2,
form C: variant peptide corresponding to a deletion of threonine 194, having a mass of 3868.1,
form D: variant peptide corresponding to a deletion of alanine 194, having a mass of 3898.1.

The abundance of the various forms of activation peptides found in the purifications of BIX, MIX and FIXwt was estimated semi-quantitatively from the spectra of FIG. 9. The results are brought together in table 3 below, in which the following nomenclature has been used:
−: absence;
(+): presence, weak intensity;
+: presence, medium intensity;
+++: presence, strong intensity;
+G=presence of monosaccharides (N-acetyl-galactose+galactose, according to the literature);
+P=peptide containing a phosphate;
+S=peptide containing a sulfate.

TABLE 3

| Peptides | BIX sample | MIX sample | FIXwt sample |
|---|---|---|---|
| A + P + S | − | +++ | − |
| B + P + S | − | + | +++ |
| B + P | − | − | +++ |
| B + S | (+) | − | − |
| A + P + S + G | − | + | − |
| B + P + S + G | − | − | (+) |
| B + G | + | − | + |
| C + G | + | − | + |
| C + P + S | − | + | − |
| D + P + S | − | +++ | − |
| A | − | − | − |
| B | +++ | − | +++ |
| C | +++ | − | +++ |
| D | − | − | − |

The mass spectrometry analysis showed great heterogeneity with regard to the various types of amino acid sequences and to the post-translational modifications of the activation peptides present in the 3 types of samples.

The Benefix® recombinant form (BIX) was shown to be composed essentially of the entity B in free form, with very little sulfation and never phosphorylated. The form C, from which amino acid T194 has been deleted, was also found in significant amount.

MIX, a purified molecule derived from plasma extracts, contained essentially the phosphorylated and sulfated form A (A+P+S) and also the form D also phosphorylated and sulfated (D+P+S).

For the FIXwt derived from the Huh7-CD4 clone, the activation peptide was also found in various forms. Only the entity B was detected in non-phosphorylated and non-sulfated form (B), in phosphorylated form (B+P), and in phosphorylated and sulfated form (B+P+S). The form C corresponding to a deletion of A194 was also found in significant amount in non-phosphorylated and non-sulfated form. The presence of a glycosylated form C(C+G) was detected, the signal showing a peptide residue partially deglycosylated during the preparation of the samples for the present study.

The mass spectrometry analyses thus showed that MIX is very predominantly composed of phosphorylated and sulfated forms. BIX was shown to be completely devoid of phosphorylation and sulfation. Consequently, the FIXwt differs from BIX by virtue of the significant presence of phosphorylated forms (+P) and of phosphorylated and sulfated forms (+P+S).

1. A recombinant human factor IX (FIX) characterized in that it is obtained by means of a preparation method comprising, or even consisting of, the steps which consist in:
causing the genetic material encoding the FIX to be expressed in vitro in a human hepatocyte cell line Huh7,
recovering the cell supernatant in which the FIX was secreted, and
optionally, purifying the synthesized FIX.

2. The recombinant human factor IX (FIX) as claimed in claim 1, characterized in that the genetic material encoding the FIX is a cDNA encoding the FIX.

3. The recombinant human factor IX (FIX) as claimed in claim 1, characterized in that the genetic material encoding the FIX is transferred into and expressed in the hepatocyte cell line by transfection.

4. The recombinant human factor IX (FIX) as claimed in claim 1, characterized in that it has a specific coagulation activity, determined by means of the technique termed activated partial thromboplastin time technique, of from 150 to 200%, preferably from 170 to 200%.

5. The recombinant human factor IX (FIX) as claimed in claim 1, characterized in that it has an electrophoretic profile identical to the plasma form of human FIX.

6. The recombinant human factor IX (FIX) as claimed in claim 1, characterized in that it is N-glycosylated and/or sialylated.

7. The recombinant human factor IX (FIX) as claimed in claim 1, characterized in that the activation peptide of said recombinant FIX is present in a phosphorylated and/or sulfated form.

8. A method for preparing a human hepatocyte cell line producing biologically active recombinant human FIX, characterized in that it comprises, or even consists of, the following steps:
causing the genetic material encoding the FIX to be expressed in vitro in said human hepatocyte cell line Huh7,
recovering the cell supernatant in which the FIX was secreted, and
optionally, purifying the synthesized FIX.

9. The preparation method as claimed in claim 8, characterized in that the genetic material encoding the FIX is a cDNA encoding the FIX.

10. The preparation method as claimed in claim 8, characterized in that the genetic material encoding the FIX is transferred into and expressed in the hepatocyte cell line by transfection.

11. A recombinant factor IX produced by the Huh7-CD4 cell line deposited and registered under number I-4234, with the CNCM [National Collection of Microorganism Cultures].

12. A cell line Huh7-CD4 deposited and registered under number I-4234, with the CNCM.
